(19) United States
(12) Patent Application Publication
(10) Pub. No.: US 2001/0056226 A1
(43) Pub. Date: Dec. 27, 2001

(54) Integrated telemedicine computer system

(76) Inventors: Richard Zodnik, San Juan Capistrano, CA (US); Gregory S. Buchert, Corona Del Mar, CA (US)

Correspondence Address: IRELL & MANELLA LLP, 840 NEWPORT CENTER DRIVE, SUITE 400, NEWPORT BEACH, CA, 92660, US

(21) Appl. No.: 09/833,933

(22) Filed: Apr. 11, 2001

Related U.S. Application Data
(60) Provisional application No. 60/198,057, filed on Apr. 18, 2000.

(51) Int. Cl. A61B 5/00

(52) U.S. Cl. 600/300; 600/549

(57) ABSTRACT

A patient medical monitor that can allow a health care provider to remotely monitor and assist a patient. The patient monitor may be coupled to a provider monitor by a network such as the Internet. The monitor may include a touch screen that displays large icons. Touching an icon may cause the monitor to transmit information to an external source. For example, touching a “HELP” icon may cause the monitor to dial an emergency phone number. The icons are large so that the patient can easily see and read the monitor. The monitor may have a plurality of output ports that can be coupled to medical sensing devices such as an electronic thermometer, pulse oximeter, stethoscope, blood pressure cuff, etc. The monitor can convert the input signals from the sensing devices into a format that can be transmitted through the network. The converted sensing input signals can be bundled with other personal data such as name, age, etc. of the patient so that the health provider can store the information and diagnostic data in a relational database

### REFERENCE TO CROSS-RELATED APPLICATIONS

This application claims priority to provisional application number No. 60/198,057 filed on Apr. 18, 2000.

### BACKGROUND OF THE INVENTION

1. Field of the Invention

The present invention relates to a patient monitor.

2. Background Information

To reduce health care cost it is generally desirable to 10% monitor and provide assistance to patients at a location remote from the health care provider. For example, it would be desirable to remotely obtain diagnostic data, such as pulse rate, temperature, skin color, etc. from a patient. Remote data retrieval would provide for health care without requiring the patient to travel to the health care provider. Additionally, remote data retrieval would allow the health care provider to monitor and assist the patient on a daily basis.

Diagnostic data is typically taken with separate devices such has a thermometer or a pulse oximeter. The data from each individual device can be transmitted to a location or more commonly, is then manually entered into a computer. The diagnostic data may then be stored in a database located within a network. The database allows medical personnel at remote locations to view the diagnostic data. Data entry of medical information is typically a time consuming process that requires trained personnel. It would be desirable if such data can be entered into a computer database directly by the patient.

Manually entering data can be challenging for patients that are not proficient at operating a computer. It would be desirable to have a single electronic unit that can be easily operated by a patient that can easily capture data from multiple diagnostic devices. It would also be desirable to provide a single electronic unit that will allow a health care provider to remotely monitor and assist a patient, taking advantage of the data obtained by the patient.

### BRIEF SUMMARY OF THE INVENTION

One embodiment of the present invention is a medical monitor that can receive information from, and transmit information to an external source. The monitor may have a touch screen that is attached to a housing and displays one or more large icons. Touching the screen may cause the monitor to transmit information to the external source.

### BRIEF DESCRIPTION OF THE DRAWINGS

FIG. 1 is a front perspective view of an embodiment of a medical monitor of the present invention;

FIG. 2 is a rear perspective view of the medical monitor;

FIG. 3a is a schematic of the medical monitor in a network.

### DETAILED DESCRIPTION OF THE PREFERRED EMBODIMENT

In general the present invention includes a patient medical monitor that can allow a health care provider to remotely monitor and assist a patient. The patient monitor may be coupled to a provider monitor by a network such as the Internet. The monitor may include a touch screen that displays large icons. Touching an icon may cause the monitor to transmit information to an external source. For example, touching a “HELP” icon may cause the monitor to dial an emergency phone number. The icons are large so that the patient can easily see and read the monitor. This will ease the use of the monitor by patients that have vision issues or are generally uncomfortable operating electronic equipment. The monitor may use voice-activated command and control to allow a patient to engage the function provided by the icons or system.

The monitor may have a plurality of input ports that can be coupled to medical sensing devices such as an electronic thermometer, pulse oximeter, stethoscope, blood pressure cuff, etc. The monitor can convert the input signals from the sensing devices into a format that can be transmitted through the network. The converted sensing input signals can be bundled with other personal data such as name, age, disease history, previous measurements using the same and/or additional devices, etc. of the patient so that the computer can store the information and diagnostic data in a relational database for review by or transmission to a health provider. The monitor may have an electronic card slot that can receive information from or write to a smart card which contains the personal information of the patient. The monitor may also have a CD slot that can receive a CD with the patient's information.

The monitors of both the health care provider and patient may have cameras to allow the creation of a two-way video conference. This allows the health care provider to view the patient and vice versa. The monitor may display messages to remind the patient to administer certain medications. The message prompts may include a picture of the medicine and verbal, video and/or written instruction on the medicine administration. The message prompts may also include instructions on obtaining diagnostic data that is then transmitted to the health care provider. This allows the health care provider to monitor the responsiveness of the patient to the medication.

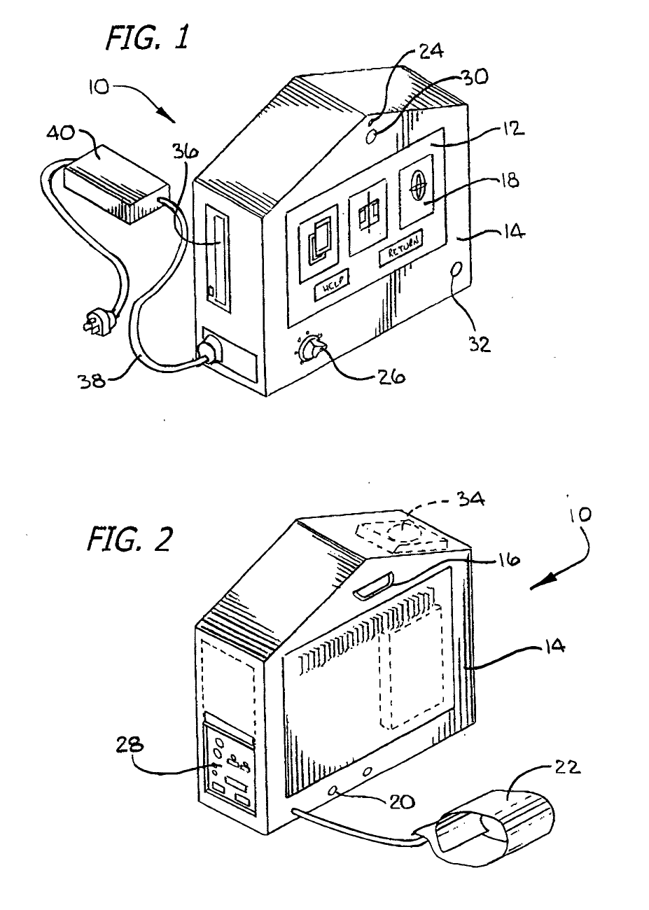

Referring to the drawings more particularly by reference numbers, FIGS. 1 and 2 show an embodiment of a patient monitor 10 of the present invention. The monitor 10 includes a screen 12 that is attached to a housing 14.

The monitor 10 may be relatively small and lightweight to allow someone to lift and move the unit 10. The housing 14 may include a handle 16 to facilitate moving the monitor 10.

The screen 12 may be a “touch” screen device that is responsive to pressure. The screen 12 may display large icons 18 which each correspond to a certain function. For example, there may be an icon that can be “touched” to cause the monitor to dial an emergency phone number such as 911. There may be an icon that can be touched by the patient to dial a health care provider, or to connect to a Web page on the World Wide Web. By way of example, the icons may be at least 3 inches high and 2 inches wide. The icons and resulting functions may create a feel of a kiosk that allows the patient to easily find information and connect to external sources.

The monitor 10 may further have a plurality of input ports 20 that can be coupled to an external device 22 such as a blood pressure cuff shown in FIG. 2. Although a blood pressure cuff is shown and described, it is to be understood that the input ports 20 can also be coupled to an electronic thermometer, a pulse oximeter, biometric scanner, stethoscope or other medical sensing devices that can sense a physical characteristic of the patient. These devices 22 can obtain diagnostic data of the patient that is stored and transmitted to an external source. Additionally, the input ports 20 may be coupled to other devices such as a keyboard, mouse, TV tuner card, barcode scanner, etc.

The monitor 10 may have a wireless receiver 24 that can be coupled to a keyboard (not shown) or other wireless device, including medical devices capable of transmitting a wireless signal. The receiver 24 can be constructed to receive infrared (IR) or radio frequency (RF) signals. The monitor 10 may further have a switch 26 (FIG. 1) that allows the operator to switch between different devices 22.

The monitor 10 may have a plurality of output ports 28. By way of example, the output ports 28 may include a printer port, audio jack, USB connector and network(s) connectors.

The monitor 10 may include a video camera 30, a microphone 32 and a speaker 34 that allows for two-way video conferences. The monitor 10 may also have a reader slot(s) 36 that can receive a smart card and/or CD (not shown) and/or handheld personal device. The smart card and/or CD and/or handheld personal device may contain personal information of the patient that is store and transmitted with diagnostic data. The monitor 10 may have a power cord 38 and a battery 40 and an uninterruptible power supply (not shown).

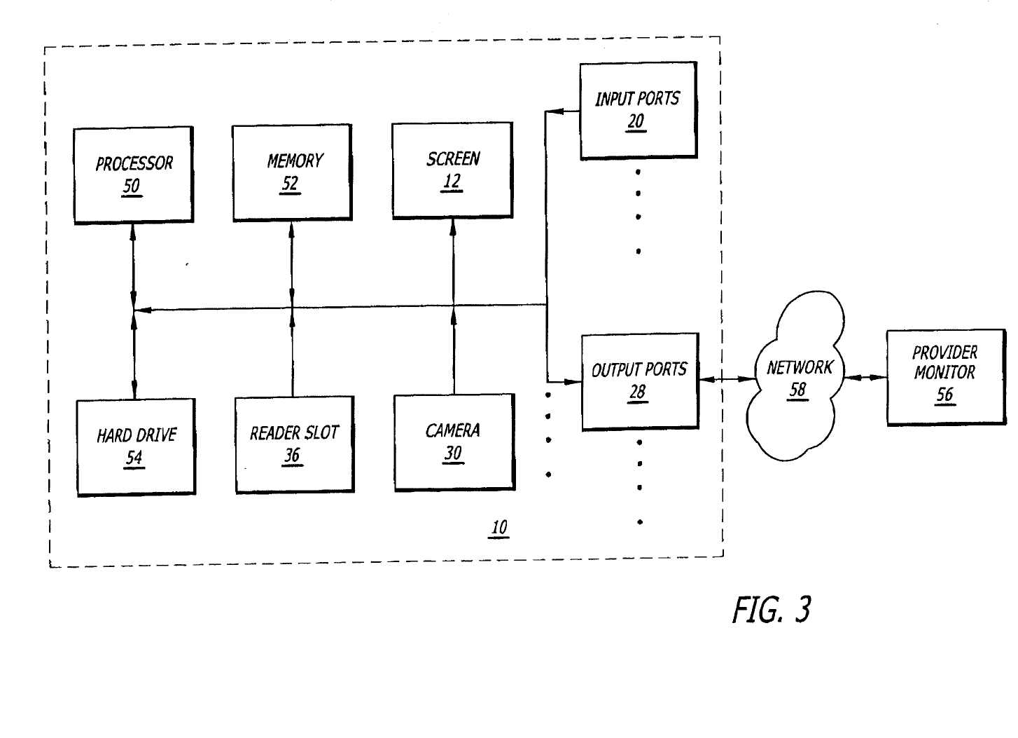

FIG. 3 shows a schematic of a monitor 10. The monitor 10 may include a processor 50 that is coupled to a memory device(s) 52. Memory 52 may include both volatile and non-volatile memory devices. The processor 50 may also be coupled to a hard disk drive 54, the reader slot 36, camera 30, screen 12, input ports 20 and output ports 28.

One or more of the output ports 28 may be connected to a provider monitor 56 by a network 58. The network may be a local area network (LAN) or a wide area network (WAN) such as the Internet that operates in accordance with Transmission Control Protocol/Internet Protocol (TCP/IP protocol). Additionally, one of the output ports 28 may be connected to a telephone network. The monitor 10 may therefore have both phone and data ports.

The provider monitor 56 may include the hardware and software found in a patient monitor 10. Additionally, the provider monitor 56 may be connected to another network such as a LAN that contains server(s), relational database(s), etc. for storing and retrieving diagnostic data and information of patients.

The processor 50 may operate in accordance with a software and/or firmware routine(s) that contain instructions and data, which together will be referred to as a software routine. The software routine may cause create messages on the screen to prompt the patient to administer medication or perform some other health information such as exercise routines. The message prompts may include graphics showing the medication, medication bottle, etc. with text relating to administering the medicine. The messages may require a response by the patient, including diagnostic information on the responsiveness to the medication. Both the messages and information can be transmitted to the provider monitor 56 for use by the health care provider. The responses(s) may be entered by touching the screen, and/or typing on a Keyboard, and/or selecting through a mouse. The software routine may convert the messages and responses into a network format such as TCP/IP.

The prompt messages may also request the patient to obtain diagnostic data through the medical sensing devices 22. Alternatively, the patient may obtain the diagnostic data without prompting from the monitor 10. The software routine of the processor 50 may convert the signals from the sensing devices into a format that can be stored into a local database or transmitted through the network 58 such as TCP/IP. The diagnostic data can also be bundled with personal information of the patient that is either entered into the memory 52 and/or 54 through a keyboard or smart card/CD or from a remote database that is accessed through a LAN or WAN. This bundle of information provides a correlation between the diagnostic data and the patient information for the health care provider. The diagnostic data and personal information is then transmitted through the network 58 to the provider monitor 56.

The software routine may provide additional graphics and text relative to health care information. Additionally, the software routine may include a browser that allows the patient to reach websites in the network 58.

The software routine may include instructions and information to establish a telephone connection with a predetermined telephone number when a certain icon is touched by the user. For example, the monitor 10 may display separate icons for “fire”, “hospital”, etc. that each cause the monitor 10 to dial the corresponding entry when depressed. The software may be scalable and upgradeable to vary the contents of a telephone number table store in memory.

The data and information is preferably encrypted before being transmitted through the network. The provider network having the appropriate software to decrypt the information and data. Additionally, the software routine may include speech recognition to accept voice commands from the user. The software may also include a speech engine so that the monitor can generate commands through the speaker 34 to the user.

The network interconnection allows the health care provider to transmit and load new and/or updated software routines to the patient monitor 10. The new and/or updated software may vary the message prompts and/or information provided by the patient monitor 10.

When used with a barcode scanner the user may scan a barcode printed on the medication container. The software routine may confirm whether the medication is appropriate for the patient through a diagnostic routine. The routine may be performed either at the patient monitor or the provider monitor. This will insure that the patient does not administer the wrong medication.

The monitor 10 may further have software and hardware to establish a two-way video conference with the provider monitor. The hardware could be provided by a video card for personal computers with the appropriate driver software.

The monitor may have varying levels of security access that can be determined by the use of passwords or biometric identifiers (e.g. fingerprint, faceprint, etc.) When physically present, a health provider may access the monitor 10 who may also use the monitor to transmit information to additional sites, such as writing an order for a medication, test or therapy.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A medical monitor that can monitor a patient and transmit information to an external source, comprising:
a housing;
a touch screen that is attached to said housing and can display a large icon;
an output port that can be coupled to the external source; and,
a controller that is coupled to said screen and said output port, said controller transmits information to the external source through said output port when the patient touches said touch screen adjacent to said large icon.

2. The monitor of claim 1, further comprising an input port that is coupled to said controller and can be coupled to one or more medical sensing devices that can sense a physical characteristic of the patient.

3. The monitor of claim 1, further comprising a camera that is attached to said housing and coupled to said output port.

4. The monitor of claim 1, wherein said controller operates in accordance with a software program that encrypts the information transmitted to the external source.

5. The monitor of said claim 2, wherein said controller converts an input signal from said medical sensing device to information in TCP/IP format.

6. The monitor of claim 1, further comprising a wireless receiver that is coupled to said controller and can be coupled to a wireless keyboard.

7. The monitor of claim 1, further comprising a card slot that is coupled to said controller and can receive an electronic card that contains personal patient information.

8. The monitor of claim 1, wherein said controller operates in accordance with a software program that prompts the patient to take a medication.

9. The monitor of claim 8, wherein the prompts include a picture and description of the medication.

10. The monitor of claim 1, wherein the information initiates a telephone call.

11. The monitor of claim 1, wherein the information includes personal patient data.

12. The monitor of claim 3, wherein the information establishes a two-way video conference.

13. A medical monitor that can be coupled to a medical sensing device, monitor a patient and transmit information to an external source, comprising:
a housing;
a screen that is attached to said housing;
an output port that can be coupled to the external source;
a camera attached to said housing and coupled to said output port;
an input port that can be coupled to the medical sensing devices; and
a controller that is coupled to said screen said input port and said output port, said controller operates in accordance with a software program that transmits information to the external source through said output port.

14. The monitor of claim 13, wherein said controller operates in accordance with a software program that encrypts the information transmitted to the external medical source.

15. The monitor of said claim 13, wherein said controller converts an input signal from said medical sensing device to information in TCP/IP format.

16. The monitor of claim 13, further comprising a wireless receiver that is coupled to said controller and can be coupled to a wireless keyboard.

17. The monitor of claim 13, further comprising a card slot that can receive an electronic card that is coupled to said controller and contains personal patient information.

18. The monitor of claim 13, wherein said controller operates in accordance with a software program that prompts the patient to take a medication.

19. The monitor of claim 18, wherein the prompts include a picture and description of the medication.

20. The monitor of claim 13, wherein the information initiates a telephone call.

21. The monitor of claim 13, wherein the information includes personal patient data.

22. The monitor of claim 3, wherein the information establishes a two-way video conference.

23. A medical network for monitoring a patient, comprising:
a patient monitor th at includes a touch screen that displays a large icon, said patient monitor transmits information in response to the patient touching said touch screen adjacent to said large icon; and,
a provider station that receives the information from said patient monitor.

24. The network of claim 23, wherein said patient monitor include input port(s) that can be coupled to a medical sensing device(s) that can sense a physical characteristic(s) of the patient.

25. The network of claim 23, wherein said patient monitor includes a camera that transmits images of the patient to said provider station.

26. The network of claim 23, wherein the information is encrypted.

27. The network of said claim 23, wherein the information is transmitted in TCP/IP format.

28. The network of claim 23, wherein said patient monitor includes a wireless receiver that is coupled to said controller and can be coupled to a wireless keyboard.

29. The network of claim 23, wherein said patient monitor includes a card slot that is coupled to said controller and can receive an electronic card that contains personal patient information.

30. The network of claim 23, wherein said patient monitor prompts the patient to take a medication.

31. The network of claim 30, wherein the prompts include a picture and description of the medication.

32. The network of claim 23, wherein the information initiates a telephone call.

33. The network of claim 23, wherein the information includes personal patient data.

34. The network of claim 25, wherein the information establishes a two-way video conference.

35. A medical network for monitoring a patient, comprising:
a medical sensing device that can sense a physical characteristic of the patient and provide an input signal; and,
a patient monitor that includes a screen that can display a video image and a camera that can capture a video image of the patient, said patient monitor being coupled to said medical sensing device to convert the input signal to a network protocol, said patient monitor transmitting the captured video image of the patient, and the converted input signal; and,
a provider station that can receive the captured video image of the patient and converted input signal transmitted by said patient monitor, said provided station having a screen that can display the received captured video signal of the patient and a camera that can capture a video image of a provider that is transmitted to said patient monitor.

36. The network of claim 35, wherein said medical sensing device is a thermometer.

37. The network of claim 36, wherein the information is encrypted.

38. The network of said claim 36, wherein the information is transmitted in TCP/IP format.

39. The network of claim 35, wherein said patient monitor includes a wireless receiver that is coupled to said controller and can be coupled to a wireless keyboard.

40. The network of claim 35, wherein said patient monitor includes a card slot that is coupled to said controller and can receive an electronic card that contains personal patient information.

41. The network of claim 35, wherein said patient monitor prompts the patient to take a medication.

42. The network of claim 41, wherein the prompts include a picture and description of the medication.

43. The network of claim 35, wherein the information initiates a telephone call.

44. The network of claim 35, wherein the information includes personal patient data.

45. A method for monitoring a patient, comprising:
pressing a touch screen adjacent to an icon; and,
transmitting information to an external source in response to the pressing of the icon.

46. The method of claim 45, wherein the information creates a telephone connection.

47. A method for monitoring a patient, comprising:
coupling a medical sensing device to a patient;
converting an input signal from the medical sensing device to information that can be transmitted in a network; and, transmitting the converted information to an external device through a network.

48. The method of claim 47, wherein patient information is attached to the converted input signal information transmitted to the external source.

49. The method of claim 47, further comprising establishing a two-way video conference between the external source and the patient.

50. A medical monitor that can be coup led to a medical sensing device, monitor a patient and transmit information to an external source, comprising:
an input port that can receive an input signal from the medical sensing device;
an output port that can be coupled to the external source; and,
a controller that is coupled to said input port and said output port, said controller converts the input signal to a network format and transmits the converted input signal and personal patient information to the external source through said output port.

51. The monitor of claim 50, further comprising a camera that is coupled to said output port.

52. The monitor of claim 50, wherein said controller operates in accordance with a software program that encrypts the information transmitted to the external source.

53. The monitor of said claim 50, wherein said controller converts the input signal from said medical sensing device to information in TCP/IP format.

54. The monitor of claim 50, further comprising a wireless receiver that is coupled to said controller and can be coupled to a wireless keyboard.

55. The monitor of claim 50, further comprising a card slot that is coupled to said controller and can receive an electronic card that contains personal patient information.

56. The monitor of claim 50, wherein said controller operates in accordance with a software program that prompts the patient to take a medication.

57. The monitor of claim 56, wherein the prompts include a picture and description of the medication.

58. The monitor of claim 50, wherein the information initiates a telephone call.

59. The monitor of claim 50, wherein the information includes personal patient data.

60. The monitor of claim 51, wherein the information establishes a two-way video conference.
